(19) United States
(12) Patent Application Publication
(10) Pub. No.: US 2023/0056593 A1
(43) Pub. Date: Feb. 23, 2023

(54) ARGININE ASPARTATE-CONTAINING COMPOSITION FOR SUPPRESSING OLD PERSON SMELL

(71) Applicant: HUMEDIX CO., LTD., Anyang-si, Gyeonggi-do (KR)

(72) Inventors: Jun Seok HWANG, Seongnam-si, Gyeonggi-do (KR); Sung Sik BANG, Hwaseong-si, Gyeonggi-do (KR)

(73) Assignee: HUMEDIX CO., LTD., Anyang-si, Gyeonggi-do (KR)

(21) Appl. No.: 17/792,844

(22) PCT Filed: Jan. 13, 2021
(86) PCT No.: PCT/KR2021/000470

§ 371 Date: Jul. 14, 2022

(30) Foreign Application Priority Data
Jan. 15, 2020 (KR) 10-2020-0005364

(51) Int. Cl. A61K 31/198; A61Q 19/02; A61P 29/00; A61Q 15/00; A61Q 19/08; A23L 33/175

(52) U.S. Cl. CPC A61K 31/198; A61Q 19/02; A61P 29/00; A61Q 15/00; A61Q 19/08; A23L 33/175; A61K 2800/782

(57) ABSTRACT

The present invention relates to a composition comprising arginine aspartate. The composition according to the present invention not only reduces 2-nonenal, but also inhibits the expression of MMP-1, has a collagen synthesis ability, reduces the expression of TNF-α, and inhibits the activity of tyrosinase. Therefore, the composition according to the present invention can be effectively used for suppressing senile body odor, alleviating wrinkles, anti-inflammation or whitening.

### TECHNICAL FIELD

The present invention relates to a composition for suppressing senile body odor comprising arginine aspartate, and more particularly, to a composition capable of suppressing senile body odor by removing 2-nonenal by comprising arginine aspartate.

### BACKGROUND ART

Body wastes accumulate in a body since the metabolic ability deteriorates due to aging of the body and thus decomposition and excretion of wastes are not smooth. A substance called 2-nonenal is one of the causes of body odor in middle- and old-aged men, and occurs in large amounts especially on the back and chest.

It is known that 2-nonenal, the aldehyde substance, rarely appears before the age of 40s, but increases rapidly as the age approaches to 40s. As people enter their 40s, omega-7 fatty acids such as 9-hexadecenoic acid increase, and these fatty acids are oxidized and decomposed to produce 2-nonenal. Omega-7 fatty acids are decomposed into 4-hydroxy-2-nonenal before the age of 40s, but with aging, decomposition into 2-nonenal increases due to an increase in omega-7 fatty acids, a decrease in metabolism, and a deterioration of antioxidation function and 2-nonenal accumulates causing senile body odor.

An example of a method to remove senile body odor includes to make the metabolism smooth through light exercise so that the wastes can be excreted well, or to wash the wastes accumulated in pores by taking a frequent shower using a vitamin C-containing product with good antioxidation function. Alternatively, it may be helpful to increase the secretion of fatty acids by eating foods rich in vitamins C and E having high antioxidation effect, or by eating meat in moderation.

Meanwhile, Korean Patent Registration No. 10-1627415 discloses a cosmetic composition capable of removing nonenal, which causes senile malodor, comprising a lysosome extract, a Japanese cypress extract, and a herbal extract. In addition, Korean Patent Laid-Open Publication No. 10-2013-0150408 discloses an antinonenal deodorant composition comprising Japanese cypress essential oil and seaweed extract.

However, such methods are difficult to effectively apply in real life or are not sufficient to remove severe senile body odor.

### DISCLOSURE

### Technical Problem

As a result of intensive research to develop a material that decomposes or removes 2-nonenal, the main cause of senile body odor, the present inventors discovered that arginine aspartate effectively removes 2-nonenal to suppress senile body odor. In addition, the inventors found that arginine aspartate has effects of wrinkle alleviation, anti-inflammation or whitening in addition to the senile body odor suppression effect, and completed the present invention.

Therefore, an object of the present invention is to provide a composition for suppressing senile body odor, alleviating wrinkles, anti-inflammation, or whitening, comprising arginine aspartate.

### Technical Solution

One embodiment of the present invention relates to a cosmetic composition for suppressing senile body odor, alleviating wrinkles, anti-inflammation, or whitening, comprising arginine aspartate.

The arginine aspartate is a salt formed of L-arginine, which is a basic amino acid, and L-aspartic acid, which is an acidic amino acid.

In one embodiment of the present invention, the arginine aspartate may be represented by the following formula (I).

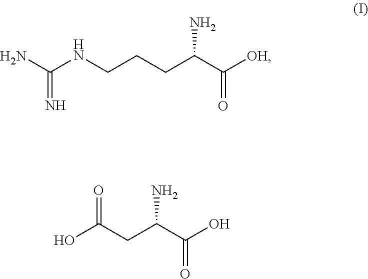 [SMILES: N=C(N)NCCC[C@H](N)C(=O)O ; N[C@@H](CC(=O)O)C(=O)O]

The arginine aspartate can be obtained commercially or can be easily prepared by methods known in the art.

It was shown that the arginine aspartate according to the present invention not only reduces 2-nonenal, but also inhibits the expression of MMP-1 (Matrix metalloproteinase-1), has a collagen synthesis ability, reduces the expression of TNF-α, and inhibits the activity of tyrosinase (Examples 1 to 5). Therefore, the arginine aspartate according to the present invention can be effectively used in a composition for suppressing senile body odor, alleviating wrinkles, anti-inflammation, or whitening, and in particular, it can be used in a composition for suppressing senile body odor that reduces 2-nonenal.

The cosmetic composition according to the present invention may comprise the arginine aspartate in an amount of about 0.001 to 10 wt %, preferably 0.01 to 5 wt % as an active ingredient. The amount of the active ingredient may be determined depending on the purpose of use.

The cosmetic composition of the present invention may comprise cosmetic ingredients usually used in the art, for example, common adjuvants such as anti-oxidant, stabilizer, solubilizer, vitamin, pigment and perfume, and carriers, in addition to the arginine aspartate.

The cosmetic composition of the present invention may be formulated as any form usually used in the art, for example, solution, suspension, emulsion, paste, gel, cream, powder, spray and the like.

In the case where the cosmetic composition is formulated as paste, cream or gel, the examples of the carriers include animal oil, vegetable oil, wax, paraffin, starch, tragacanth, cellulose derivative, polyethylene glycol, silicon, bentonite, silica, talc, zinc oxide and the like.

In the case where the cosmetic composition is formulated as powder or spray, the examples of the carriers include lactose, talc, silica, aluminum hydroxide, calcium silicate, polyamide powder and the like. In particular, in the case where the form is spray, the cosmetic composition can further comprise propellant such as chlorofluorohydrocarbon, propane/butane and dimethyl ether.

In the case where the cosmetic composition is formulated as solution or emulsion, the examples of the carriers include solvent, solubilizer and emulsifier such as water, ethanol, isopropanol, ethyl carbonate, ethyl acetate, benzyl alcohol, benzyl benzoate, propylene glycol, 1,3-butylglycol oil, glycerol fatty acid ester, polyethylene glycol, sorbitan fatty acid ester and the like.

In the case where the cosmetic composition is formulated as suspension, the examples of the carriers include liquid diluent such as water, ethanol and propylene glycol, suspending agent such as ethoxylated isostearyl alcohol, polyoxyethylene sorbitol ester and polyoxyethylene sorbitan ester, microcrystalline cellulose, aluminum metahydroxide, bentonite, agar, tragacanth and the like.

The cosmetic composition of the present invention may be applied to cosmetics such as skin, lotion, cream, essence, face pack, foundation, coloring cosmetics, sun block cream, two-way cake, face powder, compact, makeup base, makeup cover, eye shadow, lip stick, lip gloss, lip fix and eyebrow pencil.

One embodiment of the present invention relates to a pharmaceutical composition for suppressing senile body odor, alleviating wrinkles, anti-inflammation or whitening, comprising arginine aspartate.

The description of the arginine aspartate is the same as that described in the cosmetic composition.

In one embodiment of the present invention, the pharmaceutical composition may be a pharmaceutical composition for suppressing senile body odor that reduces 2-nonenal.

The pharmaceutical composition according to the present invention may be administered via oral (e.g., taking through mouth or inhalation) or parenteral (e.g., injection, transdermal absorption, intrarectal administration) routes, and the injection may be intravenous, subcutaneous, intramuscular or intraperitoneal injection. The pharmaceutical composition according to the present invention may be formulated in the form of tablet, capsule, granule, fine subtilae, powder, sublingual table, suppository, ointment, injection, emulsion, suspension, syrup, spray and the like according to the administration route. These several formulations of the pharmaceutical composition according to the present invention may be prepared using a pharmaceutically acceptable carrier which is conventionally used in the art by the known methods. Examples of the pharmaceutically acceptable carrier may include excipients, binders, disintegrating agents, lubricants, preservatives, antioxidants, isotonic agents, buffering agents, coating agents, sweeteners, solubilizers, bases, dispersing agents, wetting agents, suspending agents, stabilizers, coloring agents and the like.

Although varied depending on the types of the formulations, the pharmaceutical composition according to the present invention comprises the arginine aspartate in an amount of about 0.001 to 10 wt %, preferably 0.01 to 2 wt %.

The specific dosage of the pharmaceutical composition of the present invention may be determined depending on the kinds, weight, sex, severity of disease of mammals to be treated, including a human, the physician's decision and the like. Preferably, a daily dosage for oral administration may range from 10 to 200 mg per 1 kg of body weight. The total daily dosage may be administered in a single dose or divided doses, depending on the severity of diseases, the physician's decision and other conditions.

One embodiment of the present invention relates to a functional food for suppressing senile body odor, alleviating wrinkles, anti-inflammation or whitening, comprising arginine aspartate.

The description of the arginine aspartate is the same as that described in the cosmetic composition.

In one embodiment of the present invention, the functional food may be a functional food for suppressing senile body odor that reduces 2-nonenal.

The kinds of the functional food according to the present invention are not particularly limited. For example, the functional food may be in the form of oral preparations such as powder, granule, tablet, capsule, suspension, emulsion, syrup, or it may be added to general foods such as candies, cookies, chewing gums, ice cream, noodles, breads, beverages and the like.

The functional food of the present invention may be prepared by a conventional method according to its form, and it may suitably contain a carrier being acceptable as a food material, e.g., filling agents, extenders, binders, wetting agents, disintegrating agents, sweeteners, flavoring agents, preservatives, surfactants, lubricants, excipients and the like.

Although varied depending on the kinds of the functional food, the functional food may comprise the arginine aspartate in an amount of about 0.001 to 10 wt %, preferably 0.1 to 5 wt %.

### Advantageous Effects

The arginine aspartate according to the present invention not only reduces 2-nonenal, but also inhibits the expression of MMP-1 (Matrix metalloproteinase-1), has a collagen synthesis ability, reduces the expression of TNF-α, and inhibits the activity of tyrosinase. Therefore, the arginine aspartate according to the present invention can be effectively used in a composition for suppressing senile body odor, alleviating wrinkles, anti-inflammation or whitening.

### BRIEF DESCRIPTION OF FIGURES

FIG. 1 is a graph showing the evaluation results of the ability of the arginine aspartate to remove 2-nonenal.

### BEST MODE

The present invention will be described in more detail by following examples. It will be obvious to those skilled in the art that these examples are merely described for illustration of the present invention and the scope of the present invention is not limited thereto.

### Example 1: Evaluation of the Removal of 2-Nonenal by Arginine Aspartate

The ability of the arginine aspartate of Formula (I) according to the present invention to remove 2-nonenal was evaluated.

2-Nonenal was dissolved at a concentration of 0.1 wt % (1000 ppm) in 50 wt % ethanol aqueous solution containing 0.5 wt % Tween 80. Arginine aspartate was dissolved at a concentration of 5 wt % (50000 ppm) in a 50 wt % ethanol aqueous solution containing 0.5 wt % Tween 80.

The two solutions were mixed in a 1:1 ratio by weight to make a mixed solution. The mixed solution was left at room temperature and the change in the amount of 2-nonenal was analyzed by HPLC over time. That is, the change in the amount of 2-nonenal over time was analyzed by treating 0.05 wt % 2-nonenal with arginine aspartate at a concentration of 2.5 wt %.

Further, the 2-nonenal solution prepared above was diluted two-fold with a 50 wt % ethanol aqueous solution containing 0.5 wt % Tween 80 and the diluted solution was left at room temperature to analyze the change in the amount of 2-nonenal over time by HPLC. That is, the change in the amount of 2-nonenal over time was analyzed in a condition of 0.05 wt % of 2-nonenal untreated with arginine aspartate.

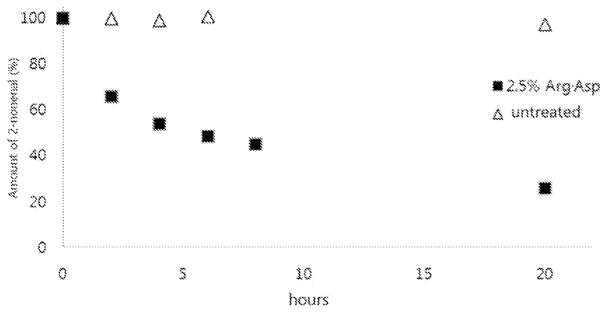

The results are shown in FIG. 1.

From FIG. 1, it was confirmed that when 2-nonenal, the causative material of body odor, was treated with arginine aspartate, the amount of 2-nonenal decreased by 74% after 20 hours, leaving only 26% by weight compared to the initial 100% by weight. On the other hand, it was confirmed that when 2-nonenal was not treated with arginine aspartate, there was no change in the amount of 2-nonenal.

### Example 2: Evaluation of the Ability of the Arginine Aspartate to Inhibit MMP-1

Human skin fibroblasts were cultured at a concentration of 1×10⁴/well. The cells were treated with arginine aspartate for each concentration, and the MMP-1 expression level was measured using a PCR device to evaluate the ability of arginine aspartate to inhibit MMP-1. A group in which the cells were not treated with the sample was used as a control group (CON), and a group in which the cells were treated with 50 ppm of adenosine was used as a positive control group. The results are shown in Table 1.

TABLE 1

MMP-1 gene expression level
(% of control)

Concentration (ppm)

| | CON | 10 | 20 | 40 |
|---|---|---|---|---|
| Arginine aspartate | 100 | 86.7 | 82.2 | 76.6 |

| | |
|---|---|
| Adenosine (50 ppm) | 73.9 |

From Table 1, it was confirmed that 40 ppm of arginine aspartate had inhibitory effect of 23.4% on MMP-1, which was similar to the MMP-1 inhibitory effect at the 50 ppm concentration of adenosine, a positive control.

### Example 3: Evaluation of Collagen Synthesis Ability

Human skin fibroblasts were cultured at a concentration of 1×10⁴/well. The cells were treated with each sample, and the amount of synthesized collagen was measured to evaluate the collagen synthesis ability. The cells were treated with adenosine at a concentration of 50 ppm as a positive control and arginine aspartate at a concentration of 10, 20 and 40 ppm. Collagen quantification was performed using a Type I Procollagen C-Peptide EIA kit (Takara, Japan). A group in which the cells were not treated with sample was used as a control group (CON). The results are shown in Table 2.

TABLE 2

| | | Procollagen gene expression level | | |
|---|---|---|---|---|
| | | (% of control) | | |
| | Concentration (ppm) | | | |
| | CON | 10 | 20 | 40 |
| Arginine aspartate | 100.0 | 123.6 | 133.0 | 136.8 |

| | |
|---|---|
| Adenosine (50 ppm) | 142.2 |

From Table 2, it was confirmed that the arginine aspartate according to the present invention exhibited an increase in collagen synthesis compared to the control group (CON). In particular, the arginine aspartate at a concentration of 40 ppm exhibited an increase in collagen synthesis similar to the case of treatment with adenosine, a positive control, at a concentration of 50 ppm.

### Example 4: Evaluation of Anti-Inflammatory Effect

HaCaT cells were seeded in a 12-well plate by 1×10⁵ cells/well, and after 24 hours, they were replaced with a basal medium and starvated for 6 hours. After that, the medium was replaced with the basal medium containing the sample and the cells were incubated for 24 hours. After removal of the medium, UVB stimulation was applied, and the basal medium was added and the cells were cultured for 24 hours. The medium was completely removed and the cells were washed twice with PBS. The cells were disrupted using TRIsure to recover RNA, and then RT-PCR was performed to amplify TNF-α. Electrophoresis was performed on an agarose gel and the expression rate was measured using the Gel Documentation system. 200 μM dexamethasone was used as a positive control. The results are shown in Table 3.

TABLE 3

| | | TNF-α gene expression level | | |
|---|---|---|---|---|
| | | (% of control) | | |
| | Concentration (ppm) | | | |
| | CON | 10 | 20 | 40 |
| Arginine aspartate | 100.0 | 76.0 | 72.9 | 72.6 |

| | |
|---|---|
| Dexamethasone (50 ppm) | 66.9 |

From Table 3, it was confirmed that arginine aspartate at a concentration of 40 ppm exhibited decrease of 37.4% in TNF-α expression level, which was similar to the TNF-α expression inhibitory effect at 200 μL dexamethasone, a positive control.

### Example 5: Evaluation of Whitening Effect

After adding 200 μL of 0.1M phosphate buffer (pH 6.8) to the Eppen tube, 20 μL of each sample solution diluted at each concentration was added. Then, 20 μL of mushroom tyrosinase (2,000 units/μL) and 200 μL of 0.3% tyrosine were added in order, and then reacted at 37° C. for 10 minutes. After 200 μL of this solution was transferred to a 96-well plate, absorbance was measured at 490 nm using an ELISA reader. 100 ppm arbutin was used as a positive control. The results are shown in Table 4.

TABLE 4

Tyrosinase activity
(% of control)

| | Concentration (ppm) | | |
|---|---|---|---|
| | CON | 10 | 100 |
| Arginine aspartate | 100.0 | 72.5 | 71.7 |
| Arbutin (100 ppm) | | 93.6 | 76.7 |

From Table 4, it was confirmed that arginine aspartate at a concentration of 100 ppm exhibited inhibition of 28.3% in tyrosinase activity, which was similar to the tyrosinase activity inhibitory effect at 100 ppm arbutin, a positive control.

1. A composition for suppressing senile body odor, alleviating wrinkles, anti-inflammation, or whitening, comprising arginine aspartate.

2. The composition for suppressing senile body odor, alleviating wrinkles, anti-inflammation, or whitening according to claim 1, wherein the arginine aspartate is represented by the following formula (I):

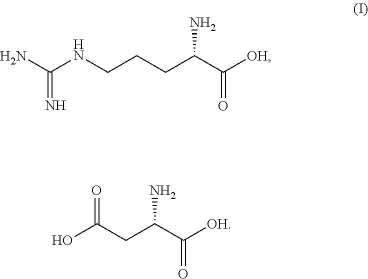 [SMILES: N=C(N)NCCC[C@H](N)C(=O)O ; N[C@@H](CC(=O)O)C(=O)O]

3. The composition for suppressing senile body odor according to claim 1, wherein the composition reduces 2-nonenal.

4. The composition according to claim 1, which is a cosmetic composition, pharmaceutical composition, or a foodstuff.

5. The composition according to claim 4, wherein the arginine aspartate is represented by the following formula (I):

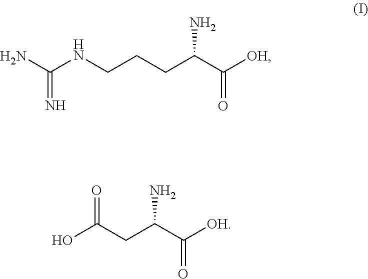 [SMILES: N=C(N)NCCC[C@H](N)C(=O)O ; N[C@@H](CC(=O)O)C(=O)O]

6. The composition according to claim 4, wherein the composition reduces 2-nonenal.

7. The composition according to claim 4, wherein the composition is a foodstuff.

8. The composition according to claim 4, wherein the composition is a cosmetic topical composition.

9. The composition according to claim 4, wherein the composition is a pharmaceutical composition.

10. A method for suppressing senile body odor, alleviating wrinkles, anti-inflammation, or whitening skin of a subject, comprising administering a composition comprising arginine aspartate to the subject.

11. The method according to claim 10, wherein the arginine aspartate is represented by the following formula (I):

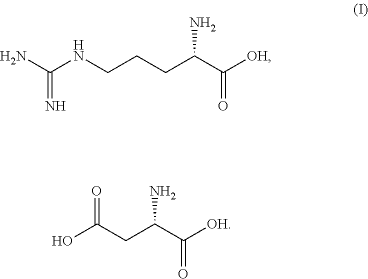 [SMILES: N=C(N)NCCC[C@H](N)C(=O)O ; N[C@@H](CC(=O)O)C(=O)O]

12. The method according to claim 10, wherein the composition reduces 2-nonenal.

13. The method according to claim 10, wherein the composition is a cosmetic composition, pharmaceutical composition, or a foodstuff.

14. A method for reducing 2-nonenal level or suppressing 2-nonenal production in a subject, comprising administering a composition comprising arginine aspartate to the subject.

15. The method according to claim 14, wherein the arginine aspartate is represented by the following formula (I):

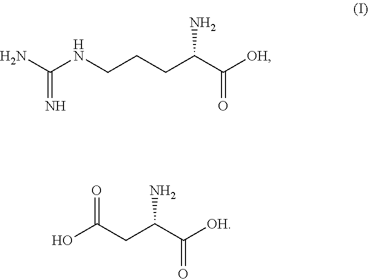 [SMILES: N=C(N)NCCC[C@H](N)C(=O)O ; N[C@@H](CC(=O)O)C(=O)O]

16. The method according to claim 14, wherein the composition is a cosmetic composition, pharmaceutical composition, or a foodstuff.
